(19) United States
(12) Patent Application Publication
(10) Pub. No.: US 2011/0146680 A1
(43) Pub. Date: Jun. 23, 2011

(54) SILICONE CATHETER CONTAINING CHLORHEXIDINE GLUCONATE

(76) Inventor: Anthony J. Conway, Chatfield, MN (US)

(21) Appl. No.: 12/822,669

(22) Filed: Jun. 24, 2010

Related U.S. Application Data
(60) Provisional application No. 61/220,384, filed on Jun. 25, 2009.

(51) Int. Cl. A61L 31/16; A61L 29/16; A61M 27/00; A61L 31/00; A61L 17/00; B05D 1/18; B05D 3/00; B05D 5/00; B23P 17/00

(52) U.S. Cl. 128/204.18; 604/265; 604/544; 604/328; 128/207.14; 604/332; 604/8; 606/231; 427/2.3; 427/2.1; 427/2.31; 427/2.28; 29/527.2

(57) ABSTRACT

The invention relates to a silicone catheter including chlorhexidine gluconate, methods of making this catheter, and methods of using it. The method of making the catheter includes contacting a silicone catheter with a liquid containing chlorhexidine gluconate. Chlorhexidine gluconate is stably incorporated into the silicone catheter.

The invention also relates to a silicone medical device or article including chlorhexidine gluconate and methods of making this such a medical device or article.

### CROSS REFERENCE TO RELATED APPLICATION

This application claims benefit of Provisional Application No. 61/220,384, filed Jun. 25, 2009, which application is incorporated herein by reference.

### FIELD OF THE INVENTION

The invention relates to a silicone catheter including chlorhexidine gluconate, methods of making this catheter, and methods of using it. The method of making the catheter includes contacting a silicone catheter with a liquid containing chlorhexidine gluconate. Chlorhexidine gluconate is stably incorporated into the silicone catheter. The invention also relates to a silicone medical device or article including chlorhexidine gluconate and methods of making this such a medical device or article.

### BACKGROUND OF THE INVENTION

Foley-type catheters are tube-like devices that are used to drain urine from a patient's bladder. Foley catheters are inserted through the urethra and typically held in place with an inflatable balloon. The balloon is in a deflated position when the catheter is first inserted. Then, once the catheter is in the proper position, the balloon is inflated with a fluid. The inflated balloon is larger in diameter than the diameter of the urethra and thereby physically inhibits movement of the catheter. Foley catheters are also known as “indwelling” catheters because they are designed to be left in place for a period of time.

In catheterization, there is a significant potential for microbial growth along the exterior surface of the catheter which can lead to serious infections such as urinary tract infections, bladder infections and the like. Such an infection can be encouraged when adjacent tissues are inflamed due to irritation from rubbing or chafing against the catheter. Such infections are undesirable and can be harmful to the patient. Undesirable microbial growth can also occur in a tube through which the catheter drains or the bag into which it drains. In addition, microbes can grow in the environs of other medical devices or articles that can be made of a silicone.

There remains a need for catheters, other articles, and methods that reduce the incidence of undesirable microbial growth associated with catheterization.

### SUMMARY OF THE INVENTION

The invention relates to a silicone catheter including chlorhexidine gluconate, methods of making this catheter, and methods of using it. The method of making the catheter includes contacting a silicone catheter with a liquid containing chlorhexidine gluconate. Chlorhexidine gluconate is stably incorporated into the silicone catheter. The invention also relates to a silicone medical device or article including chlorhexidine gluconate and methods of making this such a medical device or article.

The present invention includes a catheter including chlorhexidine gluconate. This catheter can include a catheter shaft defining a first lumen and having an outer surface. The first lumen being in fluid communication with an opening located at a distal end of the catheter shaft. The outer surface can include a silicone, the silicone includes an effective antimicrobial amount of chlorhexidine gluconate. The silicone can be pure silicone rubber. Chlorhexidine gluconate incorporated into the silicone can be free of significant amounts of chlorhexidine free base.

The present invention also includes a method of making a catheter. This method includes providing a catheter shaft defining a first lumen and an outer surface. The outer surface includes cured silicone. This method also includes immersing at least a portion of the cured silicone in a liquid containing chlorhexidine gluconate. Chlorhexidine gluconate enters and remains in the cured silicone. The catheter made by this method includes an effective antimicrobial amount of chlorhexidine gluconate.

The present invention also includes a method of catheterizing a subject. This method includes placing the present chlorhexidine gluconate containing catheter within the subject's urinary tract.

The present invention includes a medical device or article including cured silicone including an effective antimicrobial amount of chlorhexidine gluconate. At least a portion of the cured silicone can include evenly distributed, non-particulate chlorhexidine gluconate. The medical device can release an antimicrobial amount of chlorhexidine gluconate when in contact with a biological tissue or fluid.

The present invention includes a method of making a medical device. The method can include providing a medical device including cured silicone; immersing at least a portion of the cured silicone in a liquid including chlorhexidine gluconate; and producing cured silicone including an effective antimicrobial amount of chlorhexidine gluconate. The medical device of the invention can be made by the method of the invention.

### BRIEF DESCRIPTION OF THE FIGURES

FIG. 1 is a schematic view of a catheter is an original deflated configuration;

FIG. 2 is a partial cross-sectional view of an embodiment of a Foley catheter according to the present invention and including chlorhexidine gluconate;

FIG. 3 is a partial cross-sectional view of an extruded double lumen tube of the Foley catheter of FIG. 2;

FIG. 4 is a cross-sectional view of the extruded double lumen tube of FIG. 3, as shown from line 202-202′;

FIG. 5 is a partial cross-sectional view of the tube shown in FIG. 3 after an opening is formed in an outer surface;

FIG. 6 is a cross-sectional view of the tube of FIG. 5, as shown from line 204-204′;

FIG. 7 is a partial cross-sectional view of the double lumen tube shown in FIG. 5 after a portion of a capillary lumen has been filled with a polymeric bonding composition;

FIG. 8 is a cross-sectional view of the tube of FIG. 7, as shown from line 206-206′;

FIG. 9 is a partial cross-sectional view of the double lumen tube shown in FIG. 7 after a tip is affixed to a distal end of the tube;

FIG. 10 is a schematic view of a portion of a rack used to retain a plurality of tubes during manufacture of a plurality of Foley catheters;

FIG. 11 is a partial cross-sectional view of an intermediate tube similar to the tube shown in FIG. 9 at an intermediate stage of manufacture;

FIG. 12 is a partial cross-sectional view of an intermediate tube similar to that shown in FIG. 11, but following a first dipping step wherein the outer surface is coated with a bond preventing agent;

FIG. 13 is a cross-sectional view of the intermediate tube of FIG. 12, as shown from line 211-211′;

FIG. 14 is a partial cross-sectional view of an intermediate tube similar to that shown in FIG. 12, but after a subsequent dipping step or steps in which a portion of the coating of bond preventing agent has been removed;

FIG. 15 is a partial cross-sectional view of an intermediate tube similar to that shown in FIG. 14, but shown after formation of a balloon layer;

FIG. 16 is a partial cross-sectional view of an intermediate tube similar to that shown in FIG. 15, but shown after formation of a sheath layer;

FIG. 17 is a partial cross-sectional view of a portion of an embodiment of a Foley catheter having a finish layer; and

FIG. 18 is a schematic illustration of an apparatus used to automate the production of Foley catheters in accordance with the present invention.

### DETAILED DESCRIPTION OF THE INVENTION

The present invention relates to a catheter having improved antimicrobial properties. In particular, the present catheter includes chlorhexidine gluconate. Chlorhexidine gluconate can be in a silicone (e.g., silicone rubber) that forms an outer layer of the catheter. Chlorhexidine gluconate can be effective against undesirable microbes that may occur in the environs of a catheter dwelling in the urethra or bladder. It has been unexpectedly discovered that immersing cured silicone in a liquid including chlorhexidine gluconate results in the silicone absorbing chlorhexidine gluconate but not the liquid. Furthermore, the silicone absorbs and retains chlorhexidine gluconate, even when the silicone is subsequently immersed in water. Even so, the silicone catheter containing chlorhexidine gluconate reduces the growth of microbes in the surroundings of the catheter.

The present invention includes a method of making a catheter including a silicone containing chlorhexidine gluconate. The method includes immersing cured silicone in a liquid containing chlorhexidine gluconate. Suitable liquids include water. The liquid can contain, for example, about 20 wt-% chlorhexidine gluconate and about 80 wt-% water. The chlorhexidine gluconate can be in solution in the liquid. All or a portion of the catheter or cured silicone can be immersed in the liquid containing chlorhexidine gluconate. For example, the portion of the catheter that resides in the patient can be immersed in the liquid containing chlorhexidine gluconate. In an embodiment, the portion of the catheter that is manipulated outside the patient or that never enters the patient is not immersed in the liquid containing chlorhexidine gluconate.

The silicone can be immersed in the liquid containing chlorhexidine for a length of time effective to provide a catheter containing an effective antimicrobial amount of chlorhexidine gluconate. For example, the silicone can be immersed in a liquid composition of about 80 wt-% water and about 20 wt-% chlorhexidine gluconate for about 24 to about 48 hours. Generally speaking, a longer immersion time can be required for a lower concentration of chlorhexidine gluconate in liquid or for a greater thickness of absorption into the silicone.

Other suitable liquid compositions in which the catheter can be immersed include, for example, about 25 wt-% chlorhexidine gluconate and about 75 wt-% water, about 15 wt-% chlorhexidine gluconate and about 85 wt-% water, about 10 wt-% chlorhexidine gluconate and about 90 wt-% water, or about 5 wt-% chlorhexidine gluconate and about 95 wt-% water. In certain embodiments, the liquid containing chlorhexidine gluconate can be water or an aqueous composition with a minor amount of another hydroxylic or water miscible solvent, such as methanol, ethanol, acetone, dimethylformamide, dimethylsulfoxide, or the like. By minor amount is meant, for example, less than or equal to about 20 wt-%, less than or equal to about 10 wt-%, less than or equal to about 5 wt-%, less than or equal to about 2 wt-%, or less than or equal to about 1 wt-% of the liquid composition.

As used herein, the term silicone refers to a silicone or silicone rubber such as a polysiloxane, e.g., polydimethylsiloxane. A polysiloxane has the chemical formula [R₂SiO]ₙ, where R is an organic group such as methyl, ethyl, or phenyl. The silicone (e.g., silicone rubber) can be free of or substantially free of other resins or polymers. Pure silicone (e.g., silicone rubber) refers to commercially available silicones (e.g., silicone rubbers) that do not include other monomers or polymers or that include only trace or incidental amounts of other monomers or polymers. Thus, the present method can employ and the catheter can be made from or include, or an outer subject contacting portion can be made from or include, pure silicone (e.g., silicone rubber). Unexpectedly, it has been discovered that immersing non-silicone resins or polymers in the present liquid containing chlorhexidine gluconate does not result in the resin or polymer absorbing an effective antimicrobial amount of chlorhexidine gluconate.

In an embodiment, the present method includes dwelling or immersing the catheter shaft in oil before it is immersed in the liquid composition including chlorhexidine. Although not limiting to the present invention, it is believed that immersing in oil may reduce formation of biofilm on the catheter after it is in the urinary tract. Although not limiting to the present invention, it is believed that immersing in oil may increase the amount of chlorhexidine in the catheter after immersing in the liquid composition including chlorhexidine. Although not limiting to the present invention, it is believed that immersing in oil softens the catheter.

The present invention also includes a catheter made by the method of the invention. A silicone immersed in a liquid containing chlorhexidine according to the present method can include an effective antimicrobial amount of absorbed chlorhexidine gluconate.

In an embodiment, the present method of making a catheter includes providing a catheter shaft. The catheter shaft defines a first lumen and an outer surface. The first lumen is in fluid communication with an opening located at a distal end of the catheter shaft. This method also includes coating the outer surface of the catheter shaft with a silicone and curing the silicone. This method then includes immersing at least a portion of the cured silicone catheter shaft in a liquid including chlorhexidine gluconate.

In an embodiment, the present invention includes a catheter including a catheter shaft that defines a first lumen. The first lumen is in fluid communication with an opening located at a distal end of the catheter shaft. The catheter shaft has an outer surface. The outer surface includes a silicone. The silicone includes an effective antimicrobial amount of absorbed chlorhexidine gluconate. As used herein, effective antimicrobial amount refers to an amount that reduces the incidence of undesirable growth of microbes in the surroundings of the catheter in a subject's body or that reduces the population of unwanted microbes in the surroundings of the catheter in a subject's body. This reduction in incidence or population of undesirable microbes can be evidenced by inhibition shown in standard in vitro testing against common uropathogens.

The present catheter can include a balloon and the expandable surface of the balloon can include absorbed chlorhexidine gluconate. A catheter including a balloon can include a second lumen and an inflatable silicone balloon arranged in fluid communication with the second lumen. In such a catheter, the first lumen can be a fluid lumen sized to convey fluid from a patient's bladder through the catheter shaft. The second lumen can be a capillary lumen sized to transport fluid to and from the inflatable balloon to configured to inflate and deflate the balloon.

This method of making a catheter can also include other procedures required to make a complete catheter. Embodiments of such steps are described hereinbelow. For example, in an embodiment, the method also includes creating a drainage eye in an outer surface of the catheter shaft that communicates with the first lumen. In an embodiment, the catheter shaft further includes a second lumen and the catheter includes a retention balloon.

The present method also includes a method of catheterizing a subject having a urethra. This method includes placing the present catheter including chlorhexidine gluconate in the subject urinary tract, e.g. in the urethra extending into the bladder for draining the bladder. Alternatively, the catheter can be a urethral urine retention device configured to block flow of urine through the urethra and to outside the subject.

In an embodiment, chlorhexidine gluconate provides effective antimicrobial action in the environs of the catheter in the subject for about three weeks during normal use. In certain embodiments, chlorhexidine gluconate provide effective antimicrobial action in the environs of the catheter in the subject during normal use for about two weeks, about three weeks, about four weeks, about five weeks, about six weeks, about seven weeks, or about eight weeks.

The present method produces silicone that includes absorbed chlorhexidine gluconate without incorporating significant amounts of the solvent (e.g., water) in which the chlorhexidine gluconate was dissolved or dispersed. Similarly, the present silicone includes chlorhexidine gluconate absorbed evenly in the silicone. The amount of chlorhexidine gluconate can form a gradient from highest amounts at the surface of the cured silicone that was in contact with the chlorhexidine gluconate containing liquid to a lower amount of chlorhexidine gluconate at a distance from that surface in the silicone. The silicone need not, and in embodiments does not, include particulate chlorhexidine gluconate. The present silicone catheter, medical device, or medical article is not made by incorporating solid chlorhexidine gluconate in uncured or liquid silicone followed by solidifying or curing the silicone.

Unless the silicone is specifically referred to as liquid, the silicone that is immersed in chlorhexidine is cured, solid, or rubbery—not liquid. The chlorhexidine gluconate is absorbed into cured, solid, or rubbery silicone. The chlorhexidine gluconate is not mixed into liquid silicone which is subsequently cured to become solid or rubbery. Although, it is possible, in an embodiment, that chlorhexidine is added to liquid silicone that is then cured and subsequently immersed in chlorhexidine gluconate to introduce chlorhexidine gluconate into the cured, solid, or rubbery silicone.

### Antimicrobial Silicone Medical Device

In an embodiment, the present invention relates to a medical device or medical article including or being made from a silicone and having improved antimicrobial properties. In particular, the silicone includes chlorhexidine gluconate. The present invention includes any of a variety of medical devices or articles that can benefit from antimicrobial activity. For example, the present chlorhexidine containing silicone can be employed in a medical device or article that resides in a patient, that contacts a patient, that contacts an article of device that can reside in or contact a patient, that contacts fluid being introduced into or draining from a patient, or the like. Chlorhexidine gluconate can reduce the population of an undesirable microbe that can occur on or in the environs of a medical device or article. For example, a tube draining a fluid from a patient can provide a location for growth of an undesirable microbe, which can enter or be introduced into or onto the patient to cause a harmful infection of the patient. The present chlorhexidine gluconate containing silicone can reduce or eliminate the growth of the microbe on or in the tube or the biological fluid and prevent or reduce the incidence of harmful infection of the patient.

In an embodiment, the present chlorhexidine containing silicone (e.g., silicone rubber) can be on the exterior of the medical device or article. The chlorhexidine gluconate can reduce the population of microbe on or in the environs of the medical device or article. The chlorhexidine gluconate can reduce the population of microbe in a tissue or biological fluid that contacts or is near the medical device or article. In an embodiment, the present chlorhexidine containing silicone can be on the exterior of the medical device that resides in a patient.

In an embodiment, the present chlorhexidine containing silicone (e.g., silicone rubber) can be a portion of the medical device or article that contacts a biological fluid (e.g., urine, blood, sputum) or tissue. The chlorhexidine gluconate can reduce the population of microbe on the medical device or article or on or in the biological fluid or tissue. For example, a catheter, cannula, or drainage tube can be made from or can include the chlorhexidine gluconate containing silicone.

In an embodiment, the medical device or article is a ventilator tube, a drainage tube, a connector tube, or a drainage or urine bag. Ventilator tube refers to the tube that ventilates a patient on a ventilator. Medical devices or articles that can be made from or that can include the present chlorhexidine gluconate containing silicone include: a housing or overmold of a medical device, a cover or sheath for a lead, an endotracheal tube, a nasal cannula, an anchor device for drainage bag or ostomy bag, a drainage bag, an ostomy bag (e.g., a colostomy or illeostomy bag), a urine collection bag, a stoma covering, a catheter, a cannula; another type of tube, bag, receptacle, or bottle; or the like. Additional medical devices or articles that can be made from or that can include the present chlorhexidine gluconate containing silicone include: a catheter (e.g., a urinary catheter or vascular catheter, such as a peripheral or central vascular catheter), a wound drainage tube or bag, an arterial graft, a soft tissue patch, a glove, a shunt, a stent, a tracheal catheter, a wound dressing, a suture, a guide wire, or a prosthetic devices (e.g., a heart valve), an artificial organ, or the like.

The present invention includes a method of making a medical device including a silicone containing absorbed chlorhexidine gluconate. The method includes immersing the silicone of or to be employed in the medical device in a liquid containing chlorhexidine gluconate. Suitable liquids containing chlorhexidine gluconate are described above. All or a portion of the silicone of the medical device can be immersed in the liquid containing chlorhexidine gluconate. For example, the portion of the medical device that resides in or on the patient can be immersed in the liquid containing chlorhexidine gluconate. For example, in an embodiment, the portion of the medical device that is manipulated outside the patient or that never enters or contacts the patient is not immersed in the liquid containing chlorhexidine gluconate.

The silicone of the medical device can be immersed in the liquid containing chlorhexidine for a length of time effective to provide a medical device containing an effective antimicrobial amount of absorbed chlorhexidine gluconate. Suitable times and liquid compositions are described above. Suitable silicones (e.g., silicone rubber) are described above. The silicone includes an effective antimicrobial amount of absorbed chlorhexidine gluconate. As used herein, effective antimicrobial amount refers to an amount that reduces the incidence of undesirable growth of microbes in the surroundings of the medical device.

In an embodiment, the present method includes dwelling or immersing the silicone medical device in oil before it is immersed in the liquid composition including chlorhexidine. Although not limiting to the present invention, it is believed that immersing in oil may reduce formation of biofilm on the medical device when it is in contact with tissue or in a biological fluid. Although not limiting to the present invention, it is believed that immersing in oil may increase the amount of chlorhexidine in the device after immersing in the liquid composition including chlorhexidine. Although not limiting to the present invention, it is believed that immersing in oil softens the device.

### Second Antimicrobial Agent

In an embodiment, the catheter (or other medical device or article) includes chlorhexidine gluconate and a second antimicrobial agent. The second antimicrobial agent can be or include a nitrofuran (e.g., nitrofurazone). The present catheter can include any of a variety of second antimicrobial agents. The second antimicrobial agent can be effective against bacteria, fungi, viruses, or a mixture thereof. As used herein, the term “effective against” refers to reducing the incidence or occurrences of growth of the microbe (e.g., likelihood of infection), reducing the population of the microbe (e.g., killing or microbicidal activity), or reducing the growth or proliferation of the microbe (e.g., microbistatic activity). The second antimicrobial agent can be effective against microbes against which the first agent is ineffective or insufficiently effective.

The present catheter can include effective antimicrobial amounts of a nitrofuran (e.g., nitrofurazone). The amount of a nitrofuran (e.g., nitrofurazone) can be effective to inhibit growth or colonization of uropathogens that are sensitive to the compound as demonstrated by in vitro tests showing it to inhibit a broad spectrum of pathogens that can cause a urinary tract infection.

The nitrofuran antimicrobial agent can be in the form of a finely divided powder and it can have sufficient water solubility to provide effective antimicrobial action in the environs of the catheter. For example, the nitrofuran can have a mean particle size of less than or equal to about 500 microns, less than or equal to about 400 microns, less than or equal to about 300 microns, less than or equal to about 200 microns, less than or equal to about 150 microns, or less than or equal to about 100 microns. In certain embodiments, the coating on the catheter includes about 2 to about 80 wt-% nitrofuran (e.g., nitrofurazone), about 5 to about 70 wt-% nitrofuran (e.g., nitrofurazone), about 10 to about 60 wt-% nitrofuran (e.g., nitrofurazone), or about 15 to about 55 wt-% nitrofuran (e.g., nitrofurazone). Suitable nitrofurans include nitrofurantoin, nitrofurazone, nidroxyzone, nifuradene, furazolidone, furaltidone, nifuroxime, nihydrazone, nitrovin, nifurpirinol, nifurprazine, nifuraldezone, nifuratel, nifuroxazide, urfadyn, nifurtimox, triafur, nifurtoinol, nifurzide, nifurfoline, nifuroquine, mixtures thereof, and the like. Suitable nitrofurans include those that are medically acceptable for topical use, e.g., topical use on mucosal surfaces.

A dispersion or mixture of silicone rubber and nitrofurazone dispersion can be prepared as follows: 100 grams of nitrofurazone powder is wetted with approximately 10 fluid ounces of 1,1,1-trichloroethane. This mixture is agitated vigorously. In a separate container, 100 grams of uncured silicone rubber (2 parts platinum cure system, ½ part A and ½ part B (Dow Corning, Midland, Mich.)) is dispersed with about 20 grams of silicone fluid (360 fluid, 20 centistoke (Dow Corning, Midland, Mich.)) in a ratio of 5 parts to 1 in approximately 10 fluid ounces of heptane. Another 30 fluid ounces of heptane is added to the nitrofurazone/heptane mixture, and agitated continuously. Alternatively, trichloroethane, toluene, or the like can be substituted for heptane.

The nitrofurazone/heptane mixture can be passed through a filter to remove the larger nitrofurazone particles. For example, two 6-inch cone-shaped filters from TUFCO (medium mesh) are used back-to-back (one inside the other) to filter this mixture. The filtering step can be repeated three or four times to remove the larger, oversized particles of nitrofurazone which will not pass through the medium mesh TUFCO filters. When the larger particles have been removed, the nitrofurazone/trichloroethane mixture or dispersion is combined with the silicone rubber dispersion and agitated constantly. The fluid mixture of the solid nitrofurazone particles in the silicone rubber dispersion can be allowed to settle just prior to dipping to form the coating on the outer surface of the catheter.

In an embodiment, the coating that contains antimicrobial agent also includes silicone fluid. This silicone fluid can provide for more rapid diffusion of the antibacterial agent upon exposure to aqueous medium; can provide a softer, more pliable device; can provide a smoother outer surface of the catheter; or a combination thereof.

### Other Chlorhexidine Salts

In an embodiment, the catheter or medical device includes a chlorhexidine salt other than chlorhexidine gluconate. Other chlorhexidine salts that can be employed in such an embodiment include chlorhexidine diphosphanilate, chlorhexidine digluconate, chlorhexidine diacetate, chlorhexidine dihydrochloride, chlorhexidine dichloride, chlorhexidine dihydroiodide, chlorhexidine diperchlorate, chlorhexidine dinitrate, chlorhexidine sulfate, chlorhexidine sulfite, chlorhexidine thiosulfate, chlorhexidine di-acid phosphate, chlorhexidine difluorophosphate, chlorhexidine diformate, chlorhexidine dipropionate, chlorhexidine di-iodobutyrate, chlorhexidine di-n-valerate, chlorhexidine dicaproate, chlorhexidine malonate, chlorhexidine succinate, chlorhexidine succinamate, chlorhexidine malate, chlorhexidine tartrate, chlorhexidine dimonoglycolate, chlorhexidine mono-diglycolate, chlorhexidine dilactate, chlorhexidine di-α-hydroxyisobutyrate, chlorhexidine diglucoheptonate, chlorhexidine di-isothionate, chlorhexidine dibenzoate, chlorhexidine dicinnamate, chlorhexidine dimandelate, chlorhexidine di-isophthalate, chlorhexidine isoethionate chlorhexidine di-2-hydroxy-napthoate, and chlorhexidine embonate. In an embodiment, the other chlorhexidine salt includes chlorhexidine acetate, chlorhexidine formate, chlorhexidine gluconate, chlorhexidine hydrochloride, chlorhexidine isoethionate, chlorhexidine lactate, or chlorhexidine succinamate.

### Illustrated Embodiments

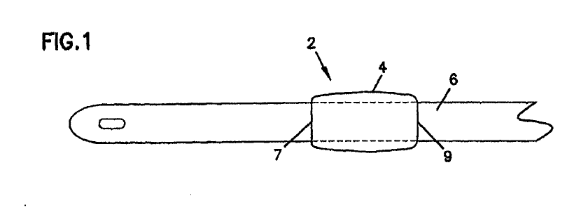

FIG. 1 shows a schematic view of a catheter according to the present invention including catheter shaft 6 and optional balloon 4, in a deflated configuration 2.

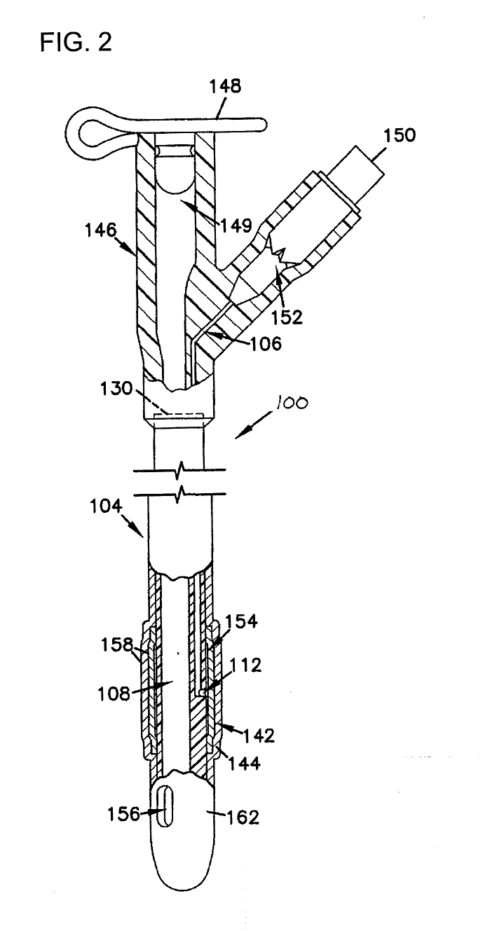

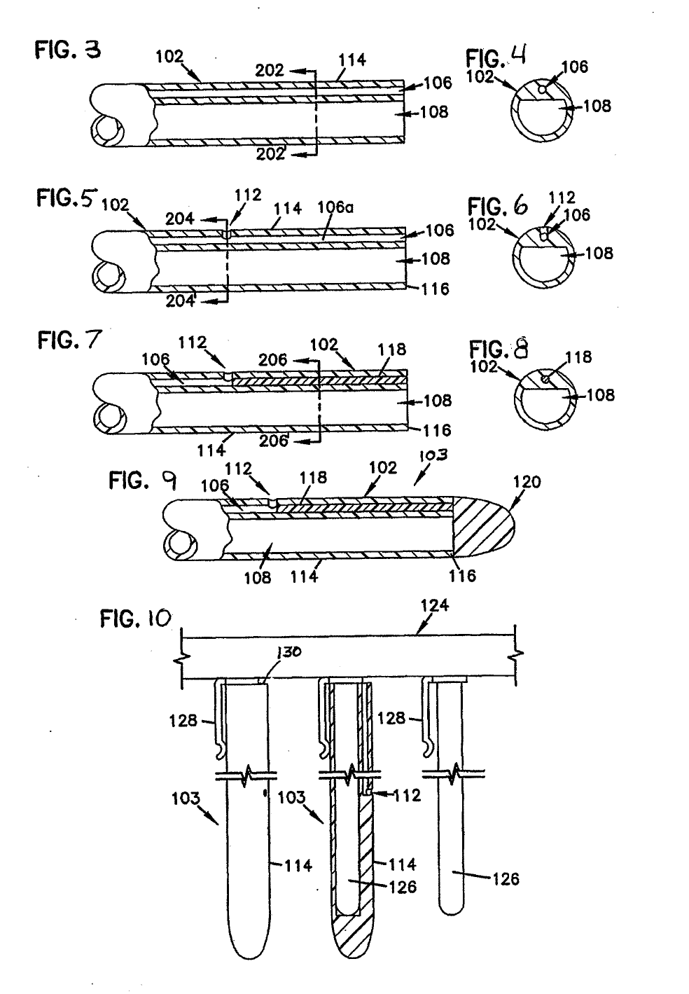

Referring now to FIG. 2, one embodiment of a Foley catheter 100 according to the present disclosure is illustrated. The Foley catheter 100 includes a catheter shaft 104 and an end piece 146. The catheter shaft 104 includes optional retention balloon 158 having a balloon cavity 154.

Referring now to FIG. 4, the catheter shaft 104 (FIG. 2) of the catheter 100 is constructed from a double lumen tube 102 (which, in certain embodiments, can be or form a catheter shaft). The double lumen tube 102 is typically extruded, however, the double lumen tube can be made by any known process that yields a double lumen tube construction. The double lumen tube 102 defines a capillary lumen 106 and a fluid conduit lumen 108. Typically, the double lumen tube 102 is made of a resilient polymeric material. In one embodiment, the polymeric material is a biocompatible polymeric material, such as silicone rubber, for example.

The double lumen tube 102 is cut to a desired length. Referring to FIGS. 5 and 6, a capillary lumen access opening 112 is created in an outer surface 114 of the double lumen tube 102. The capillary lumen access opening 112 communicates with the capillary lumen 106.

Referring now to FIGS. 7-9, an intermediate tube 103 (FIG. 9) is prepared from the double lumen tube 102 shown in FIG. 5. In preparing the intermediate tube 103, a measured amount of a filling composition or polymeric bonding composition 118, such as silicone rubber or another suitable polymeric bonding material, is injected into a portion 106a (FIG. 5) the capillary lumen 106 from a distal end 116 of the double lumen tube 102. The capillary lumen portion 106a is filled with the filling composition 118 up to a point just below the capillary lumen access opening 112.

A tip 120, such as a rounded silicone rubber tip, is affixed to the distal end 116 of the tube 102. One method of affixing the tip 120 to the tube 102 includes inserting the distal end 116 of the tube 102 into a molding apparatus (not shown) to mold the tip 120 on the end of the tube 102. Other methods of affixing the tip 120 can be employed.

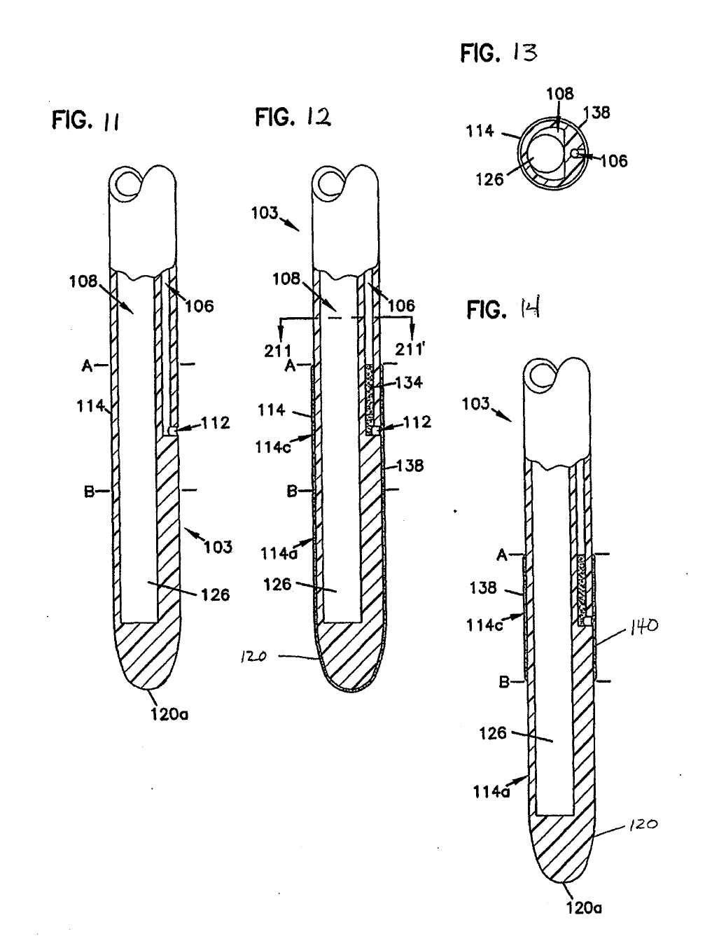

In one embodiment of the present method, the intermediate tube 103 (FIG. 9) is made entirely of silicone rubber. For example, the tip 120 and the filling composition 118 of the intermediate tube 103 are of the same material (silicone rubber) as the double lumen tube 102. Therefore, the tip 120 and the filling composition 118 form integral portions of the intermediate tube 103. FIGS. 11-16 show the intermediate tube 103 as an integral polymeric unit made of a single material.

Preferably the process of manufacturing the catheter 100 is an automated process. One of skill in the art will appreciate that while the methods are described as practiced in an automated fashion, the methods can also be practiced in a non-automated or manual, hand-performed fashion, or a semi-automated fashion.

The automated process involves securing a plurality of the intermediate tubes 103 to a rack or pallet 124, as shown in FIG. 10. The pallet 124 includes a plurality of support rods 126 so that entire sets of catheters 100 can be manufactured simultaneously. In one embodiment, the pallet 124 has 400 spring steel support rods 126 attached to the pallet 124 in a 20-by-20 configuration. Each of the rods 126 is about 1 inch from adjacent rods.

Referring still to FIG. 10, each of the support rods 126 is equipped with a retaining clip 128. The intermediate tubes 103 are secured on the support rods 126 by positioning the individual support rods 126 within the fluid conduit lumens 108 (FIG. 9) of the intermediate tubes, and sliding the intermediate tubes 103 up over the support rods 126. Each of the intermediate tubes 103 is typically positioned on the support rod 126 such that a proximal end 130 of the intermediate tube 103 abuts against the base of the retaining clips 128, or such that the tip 120 of the intermediate tube 103 fits snugly against the distal tip of the support rod 126. Although not shown, it is believed that the intermediate tubes 103 can be secured on the support rods 126 without the aid of the retaining clips 128. This is because extruded double lumen tubes 102 generally have a slight bend. This permits the intermediate tube 103 to be secured on the support rod 126 via a friction fit without the aid of the clip 128.

FIG. 18 schematically illustrates the pallet 124 loaded with the plurality of intermediate tubes 103. The pallet 124 transfers the intermediate tubes 103 from place to place via a transporting mechanism 122. For example, the transporting mechanism 122 moves or transfers the loaded pallet 124 between a series of baths or dip tanks used to manufacture the completed Foley catheter 100 shown in FIG. 2. The series of dip tanks are used to form the catheter shaft 104 having the retention balloon 158 of the Foley catheter 100.

In particular, after the intermediate tubes 103 loaded on the pallet 124, the intermediate tubes 103 are transported to a first bath or dip tank 133 by the transporting mechanism 122 (FIG. 18). The first dip tank 133 is raised so that all of the intermediate tubes 103 are simultaneously coated with a bond preventing agent; preferably, a removable bond preventing agent. While the present method relates to machinery that raises and lowers the dip tanks relative to the pallet 124, it is contemplated that the pallet 124 can also be lowered and raised relative the dip tanks.

Still referring to FIG. 18, the intermediate tubes 103 are immersed or dipped into the first dip tank 133 containing the bath of the removable bond preventing agent. The removable bond preventing agent includes materials that form a semi-solid film or coating on surfaces when cooled or dried. Examples of such materials include petroleum jelly or petrolatum, other oil base substances that form a semi-solid film upon cooling to room temperature, liquid soaps that dry to form a semi-solid film, aqueous soap or detergent solutions, aqueous or oil based film forming materials, and the like. In one method, hot petrolatum is used, and in another method, a liquid soap, such as LIQUID IVORY® soap from Proctor & Gamble, Cincinnati, Ohio, is used.

Referring now to FIG. 12, the intermediate tubes 103 are immersed in the first dip tank 133 to a desired level designated by line A. Immersing the intermediate tubes 103 into the first bath 133 coats the outer surface 114 of the intermediate tube 103 with the removable bond preventing agent. In addition, the agent enters the capillary lumen access opening 112 and runs up into the capillary lumen 106 (as shown in FIG. 12). In one embodiment the agent is petrolatum, heated to about 140°-160° F., typically about 150° F. At this temperature, the petrolatum runs up into the capillary lumen 106 through the capillary lumen access opening 112 with the assistance of the “capillary effect”, which draws the fluid into the capillary lumen 106 to the level 133a (FIG. 18) of the petrolatum in the first dip tank 133. As the intermediate tubes 103 are withdrawn from the hot petrolatum, petrolatum on each of the tubes 103 cools and solidifies to form a semi-solid bond preventing coating 138 (FIG. 12) on the outer surface 114. Likewise, a semi-solid filling 134 in the capillary lumen 106 and the capillary lumen access opening 112 is created, which cooperate to plug the capillary lumen access opening 112.

In an alternate embodiment, the bond preventing agent in the first dip tank 133 is liquid soap. The liquid soap is typically at a room temperature (about 62°-74° F.). When the tubes 103 are withdrawn from the first dip tank of liquid soap, the soap dries to form the bond preventing coating 138, just as the hot petrolatum did when cooled. Although both of these bond preventing agents are effective, there is some advantage to using liquid soap. Liquid soap does not require the added expense of providing a heated dip tank. Further, in certain embodiments, soap is easier to remove from the capillary lumen 106 and the subsequently formed balloon cavity 154 (FIG. 2).

After the outer surface 114 of the intermediate tubes 103 is coated and the capillary lumen 106 and the capillary lumen access openings 112 are plugged with the bond preventing agent, the intermediate tubes 103 are dipped in a series of dip tanks provided to remove a portion of the bond preventing coating 138. As shown in FIGS. 12 and 14, the coating 138 is removed from a portion 114a of the outer surface 114 below the line designated B. In one method, for example, the step of removing the portion of bond preventing coating 138 includes dipping the intermediate tubes 103 in series of different dip tanks.

In particular, one method includes advancing and positioning the pallet 124 at a second dip tank 135 (FIG. 18) containing white USP petrolatum heated to about 250° F. The intermediate tubes 103 are immersed into the super-heated petrolatum to a level designated by line B in FIGS. 12 and 14. The super-heated petrolatum contacts the coating 138 on outer surface 114 of the intermediate tubes 103 to largely remove the coating 138 from the outer surface portion 114a of the intermediate tubes 103. The bond preventing coating 138 is removed from a location where the distal end of the retention balloon 158 will be located (designated by line B) to the distal end 120a of the tip 120 of the intermediate tubes 103. Some residual petrolatum may remain on the outer surface portion 114a; however, most of the petrolatum is removed.

Referring to FIG. 18, the pallet 124 then advances to a third dip tank 137 containing mineral spirits heated to about 200° F. The intermediate tubes 103 are immerse into the mineral spirits to the same depth as they were immersed in the super-heated petrolatum in the second dip tank 135. The mineral spirits remove all but a trace amount of the bond preventing coating 138 from the outer surface portion 114a of the intermediate tube 103.

Last, the pallet 124 moves to a fourth dip tank 139 containing a volatile organic solvent such as toluene, trichloroethane or the like. The intermediate tubes 103 are immersed in the fourth tank 139 to the same depth as previously immersed in the second and third tanks 135 and 137. The organic solvent removes essentially all traces of the coating 138 from the outer surface portion 114a of the intermediate tube 103. As shown in FIG. 14, the intermediate tube 103 now has a band 140 of the bone preventing coating 138 located around the axial circumference of the intermediate tube 103. The band 140 is located along a portion 114c of the outer surface 114 where the retention balloon 158 and the balloon cavity 154 are subsequently formed.

After the outer surface portion 114a of the intermediate tube 103 is substantially stripped of the bond preventing coating 138, the intermediate tubes 103 are dipped in a polymeric bonding composition, such as silicone rubber. In one method, the pallet 124 advances to a fifth dip tank 141 containing a heptane dispersed solution of silicone rubber (such as Dow Corning C6-515 or another appropriate balloon compound).

The intermediate tubes 103 are immersed in the fifth dip tank 141 so that the silicone rubber covers and extends the length of intermediate tube 103 up to line C shown in FIG. 15. In some embodiments, line C is about 0.25 inches above the top of the band 140 of the bond preventing coating 138. This deposition process can be repeated until a balloon layer 142 having a desired diameter relative to a predetermined diameter of the catheter shaft 104 is formed. This silicone rubber can include the second antimicrobial agent.

As shown in FIG. 15, the balloon layer 142 does not extend along the entire length of the intermediate tube 103. Rather, the intermediate tubes 103 are dipped in a solvent to remove a portion of the silicone rubber located below line D of FIG. 15. In some embodiments, line D is about 0.25 inches below the band 140 of bond preventing coating 138. The resulting layer is the balloon layer 142 of the Foley catheter 100. Referring to FIG. 18, removing the portion of silicone rubber involves advancing the pallet 124 to a sixth dip tank 143 containing a solvent effective to remove the deposited silicone rubber. Suitable solvents include xylene or toluene.

At this point, the intermediate tubes 103 can be air dried for approximately 30 minutes to remove or evaporate solvents from the balloon layer 142. In addition, the balloon layer 142 of the tubes 103 can be cured before further processing; however, in some methods, the curing can be delayed until later in the processing. One of skill in the art will appreciate that there are many methods of curing silicone rubber. By way of example, the silicone rubber can be cured through a heat cure step for approximately two hours at a temperature just below the boiling point of any solvent used in any of the silicone rubber dip solutions.

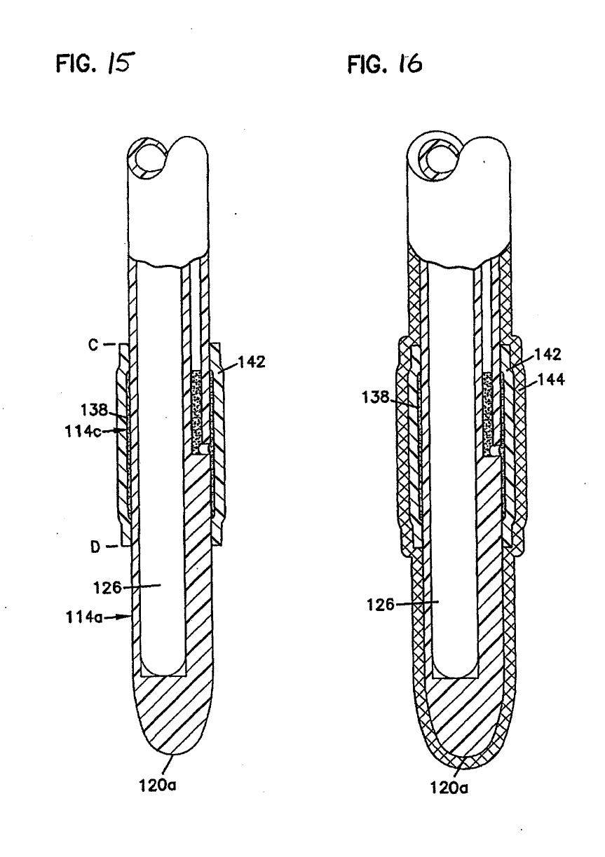

Referring now to FIG. 16, after the balloon layer 142 has been formed, a substantial majority of the intermediate tube 103 is immersed into a solution (e.g. heptane dispersed solution) of silicone rubber (such as Dow Corning C6-515 or another appropriate balloon compound) to form a sheath layer 144. This silicone rubber can include the second antimicrobial agent. In particular, the pallet 124 moves to a seventh dip tank 145 (FIG. 18) containing the solution of silicone rubber. The intermediate tubes 103 are immersed into the seventh tank 145 as many times as is necessary to obtain the desired sheath layer thickness. The sheath layer 144 is then allowed to air dry for a period of about 30 minutes.

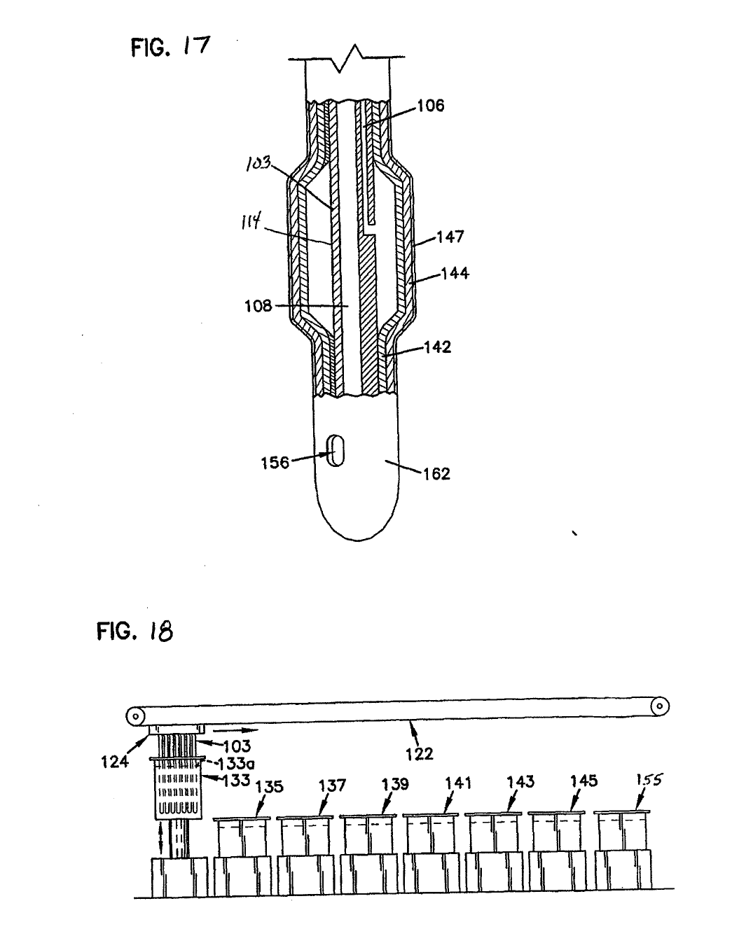

Optionally, the pallet 124 can be advanced to an eighth dip tank (not shown) containing a thin finish-type silicone rubber (such as Dow Corning 4720). This silicone rubber can include the second antimicrobial agent. The intermediate tubes 103 are dipped in the finish-type silicone rubber to create a finish layer 147 (FIG. 17). The finish layer 147 provides beneficial tactile properties to the exterior of the catheter shaft 104 of the Foley catheter 100.

The balloon layer 142, the sheath layer 144, and the optional finish layer 147 formed on the intermediate tube 103 now define the catheter shaft 104. The catheter shaft 104 is typically allowed to air dry to permit solvents in the balloon layer 142 and the sheath layer 144 to evaporate. Typically, the shaft 104 is dried, and subsequently cured, at an elevated temperature. In one method, the catheter shafts 104 are permitted to dry for approximately two hours, and then are heat cured for an additional two hours. The heat curing process includes exposing the catheter shafts 104 to a temperature chamber at about 200° F. Care is taken to keep the curing temperature below the boiling temperatures of the solvent so as to prevent unsightly bubbling of the solvent within the balloon layer 142 and the sheath layer 144. One of skill in the art will appreciate that the drying time and the curing time and temperature are approximate and can be varied depending on the specific materials and solvents used.

Optionally, after the catheter shaft 104 is dried, cured, and cooled (but before it is immersed in a liquid composition (e.g., water) including chlorhexidine gluconate), the catheter shaft 104 is immersed in oil. Referring to FIG. 19, in the oil-dwelling manufacturing step, the pallet 124 moves to a tenth dip tank (not shown) containing oil. In one method, the oil is mineral oil, such as Holland Drake Oil No. 7 or No. 9, for example. The catheter shaft 104 soaks or dwells within the tank 155 of oil for a period of time, up to 72 hours, for example, about 24 hours or about 12 hours. Typically, the oil is at room temperature. In alternative methods, the oil can be heated, for example, to about 200° F., to speed up the absorption of oil and reduce the dwell time. Other types of oils and other immersion periods can be employed to add oil to the silicone catheter.

After the catheter shaft 104 is dried, cured, and cooled (and, optionally, immersed in oil), the catheter shaft 104 can be immersed in a liquid composition (e.g., water) including chlorhexidine gluconate. One feature of the present disclosure relates to the method of manufacturing the disclosed Foley catheter 100, including the step of dwelling or immersing the catheter shaft 104 in a liquid composition including chlorhexidine gluconate. Referring to FIG. 18, in the dwelling manufacturing step, the pallet 124 moves to a ninth dip tank 155 containing a liquid composition including chlorhexidine gluconate. The catheter shaft 104 soaks or dwells within the tank 155 of this composition for a period of time, up to 4 hours, typically about 2 hours. Typically, this composition is at room temperature. In alternative methods, this composition can be heated, for example, to about 120° F., to speed up the absorption of the second antimicrobial agent and reduce the dwell time. Other types of compositions and other immersion periods can be employed to impregnate the silicone catheter construction with chlorhexidine gluconate.

To complete the Foley catheter 100 as shown in FIG. 2, the end piece 146 is secured to the proximal end 130 of the catheter shaft 104. The end piece 146 can include a cap 148 for closing a first proximal opening 149 to the fluid conduit lumen 108. In the illustrated embodiment, the end piece 146 is equipped with a luer valve 150 for engagement in and closure of a second proximal opening 152 communicating with the capillary lumen 106. The completed Foley catheter 100 also includes a drainage eye or fluid conduit access opening 156 formed in an exterior surface 162 of the catheter shaft 104. The drainage eye 156 is in fluid communication with the fluid conduit lumen 108.

In one method of manufacture, the end piece 146 is made by a process of injection molding. In particular, the proximal end 130 of the balloon catheter shaft 104 is inserted into an injection molding apparatus after the balloon layer 142 and the sheath layer 144 have been cured. A polymeric bonding composition, such as silicone rubber, is then injected into the mold (not shown) and the end piece 146 is molded onto the proximal end 130 of the balloon catheter shaft 104 to make the completed Foley catheter 100 shown in FIG. 2.

In an alternative method, the end piece 146 is molded to the proximal end 130 of the double lumen tube 102 prior to the automated process of immersing the intermediate tube 103. In this alternative method, the double lumen tube 102 is inserted into the injection molding apparatus, the polymeric bonding composition is then injected into the mold, and the end piece 146 is molded onto the double lumen tube 102. The intermediate tube 103 is then constructed. Subsequently, the first proximal opening 149 of the end piece 146 is secured to the support rod 126 by the retaining clip 128. The intermediate tube 103 is then dipped in the series of baths or dip tanks as previously described.

Referring now to FIG. 17, the retention balloon 158 of the Foley catheter 100, which includes the balloon layer 142 and the sheath layer 144, does not bond to the outer surface 114 of the intermediate tube 103. The retention balloon 158 is free to expand or inflate due to the bond preventing coating 138 that remained on the outer surface portion 114c (FIGS. 12 and 14) of the intermediate tube 103 during manufacture.

When a fluid is pumped or injected into the capillary access lumen 106 of the Foley catheter 100, the retention balloon 158 and the balloon cavity 154 expand. Any of a variety of known tests can be used to ensure that there are no leaks in the retention balloon 158 of the Foley catheter 100. Typically, a hot aqueous solution is used to test for leaks in the retention balloon 158. The hot aqueous solution also functions to remove the remaining bond preventing coating 138 and filling 134 (FIG. 12) from the balloon cavity 154 and the capillary lumen 106 respectively.

While the present method of manufacturing has been described in the making of a silicone rubber catheter, it is contemplated that the principles of the disclosed method can also be used in the making of a latex catheter. Further, although the present description relates to the making of a silicone rubber catheter, the principles disclosed can also be applied to the making of other silicone rubber devices, such as gastrostomy and other feeding tube devices, suprapubic catheters, and enema cuffs, for example.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

It should also be noted that, as used in this specification and the appended claims, the term “configured” describes a system, apparatus, or other structure that is constructed or configured to perform a particular task or adopt a particular configuration. The term “configured” can be used interchangeably with other similar phrases such as arranged and configured, constructed and arranged, adapted and configured, adapted, constructed, manufactured and arranged, and the like.

All publications and patent applications in this specification are indicative of the level of ordinary skill in the art to which this invention pertains.

The invention has been described with reference to various specific and preferred embodiments and techniques. However, it should be understood that many variations and modifications may be made while remaining within the spirit and scope of the invention.

We claim:

1. A method of making an antimicrobial catheter comprising:
providing a catheter; the catheter comprising a catheter shaft defining a first lumen and having an outer surface; the first lumen being in fluid communication with an opening located at a distal end of the catheter shaft; the outer surface comprising a silicone;
immersing at least a portion of the silicone in a liquid comprising chlorhexidine gluconate to produce a silicone comprising an effective antimicrobial amount of chlorhexidine gluconate.

2. The method of claim 1, wherein the silicone comprises about 0.05 to about 5 wt-% chlorhexidine gluconate.

3. The method of claim 1, wherein immersing comprises immersing in water comprising chlorhexidine gluconate.

4. The method of claim 3, comprising immersing in a liquid composition comprising about 20 wt-% chlorhexidine and about 80 wt-% water.

5. The method of claim 1, comprising immersing for about 24 to about 48 hours.

6. The method of claim 1, wherein the catheter shaft further comprises a second lumen and the catheter comprises a retention balloon.

7. The method of claim 1, further comprising creating a drainage eye in an outer surface of the catheter shaft that communicates with the first lumen.

8. The method of claim 1, wherein the silicone further comprises particulate nitrofurazone antimicrobial agent.

9. The method of claim 8, wherein providing the catheter comprises:
providing the catheter shaft;
coating the outer surface of the catheter shaft with an uncured silicone comprising particulate nitrofurazone antimicrobial agent; and
curing the silicone comprising particulate nitrofurazone antimicrobial agent.

10. The method of claim 1, wherein providing the catheter comprises:
providing the catheter shaft;
coating the outer surface of the catheter shaft with an uncured silicone; and
curing the silicone.

11. A catheter comprising:
a catheter shaft defining a first lumen and having an outer surface, the first lumen being in fluid communication with an opening located at a distal end of the catheter shaft; and
the outer surface comprising a silicone, the silicone comprising an effective antimicrobial amount of absorbed chlorhexidine gluconate.

12. The catheter of claim 11, further comprising a second lumen and an inflatable silicone balloon arranged in fluid communication with the second lumen.

13. The catheter of claim 12, wherein the first lumen is a fluid lumen sized to convey fluid from a patient's bladder through the catheter shaft; and
the second lumen is a capillary lumen sized to transport fluid to and from the inflatable balloon to inflate and deflate the balloon.

14. The catheter of claim 11, wherein the silicone further comprises particulate nitrofurazone antimicrobial agent.

15. The catheter of claim 11, wherein the catheter is made by a process comprising:
providing a tube defining a first lumen and an outer surface, the first lumen being in fluid communication with an opening located at a distal end of the tube;
coating the outer surface of the tube with an uncured silicone, the silicone;
curing the silicone;
immersing at least a portion of the cured coated silicone tube in a liquid comprising chlorhexidine gluconate.

16. The catheter of claim 15, wherein coating comprises coating the outer surface of the tube with an uncured silicone comprising particulate nitrofurazone antimicrobial agent.

17. A method of catheterizing a subject, the subject having a urethra, the method comprising:
providing a catheter, the catheter shaft defining a first lumen and having an outer surface, the first lumen being in fluid communication with an opening located at a distal end of the catheter shaft; the outer surface comprising a silicone, the silicone comprising an effective antimicrobial amount of chlorhexidine gluconate; and
placing the catheter within the subject's urinary tract.

18. A method of making a medical device comprising:
providing a medical device comprising cured silicone;
immersing at least a portion of the cured silicone in a liquid comprising chlorhexidine gluconate;
producing cured silicone comprising an effective antimicrobial amount of chlorhexidine gluconate.

19. The method of claim 18, wherein the silicone comprises about 0.05 to about 5 wt-% chlorhexidine gluconate.

20. The method of claim 18, wherein immersing comprises immersing in water comprising chlorhexidine gluconate.

21. The method of claim 20, comprising immersing in a liquid composition comprising about 20 wt-% chlorhexidine and about 80 wt-% water.

22. The method of claim 18, comprising immersing for about 24 to about 48 hours.

23. The method of claim 18, wherein the medical device is a ventilator tube, a drainage tube, a connector tube, or a drainage bag.

24. The method of claim 23, wherein the medical device is a tube leading from a catheter to a bag.

25. The method of claim 18, wherein the medical device is a housing or overmold of a medical device, a cover or sheath for a lead, an endotracheal tube, a nasal cannula, an anchor device for drainage bag or ostomy bag, a drainage bag, an ostomy bag, a urine collection bag, a stoma covering, a catheter, or a cannula.

26. The method of claim 18, wherein the medical device is a catheter, a wound drainage tube or bag, an arterial graft, a soft tissue patch, a glove, a shunt, a stent, a tracheal catheter, a wound dressing, a suture, a guide wire, or a prosthetic device.

27. A medical device comprising cured silicone, at least a portion of the cured silicone comprising absorbed chlorhexidine gluconate,
wherein the medical device releases an antimicrobial amount of chlorhexidine gluconate when in contact with biological tissue or fluid.

28. The medical device of claim 27, wherein the device is made by a process comprising:
providing the medical device comprising cured silicone;
immersing at least a portion of the cured silicone in a liquid comprising chlorhexidine gluconate;
producing cured silicone comprising an effective antimicrobial amount of chlorhexidine gluconate.

29. The medical device of claim 27, wherein the medical device is a ventilator tube, a drainage tube, a connector tube, or a drainage bag.

30. The medical device of claim 29, wherein the medical device is a tube leading from a catheter to a bag.

31. The medical device of claim 27, wherein the medical device is a housing or overmold of a medical device, a cover or sheath for a lead, an endotracheal tube, a nasal cannula, an anchor device for drainage bag or ostomy bag, a drainage bag, an ostomy bag, a urine collection bag, a stoma covering, a catheter, or a cannula.

32. The medical device of claim 27, wherein the medical device is a catheter, a wound drainage tube or bag, an arterial graft, a soft tissue patch, a glove, a shunt, a stent, a tracheal catheter, a wound dressing, a suture, a guide wire, or a prosthetic device.

33. The method of claim 1, further comprising, before immersing in a liquid comprising chlorhexidine, immersing a least a portion of the silicone in oil.

34. The method of claim 18, further comprising, before immersing in a liquid comprising chlorhexidine, immersing a least a portion of the silicone in oil.
